(19) United States
(12) Patent Application Publication
(10) Pub. No.: US 2015/0245952 A1
(43) Pub. Date: Sep. 3, 2015

(54) METHOD OF FABRICATING A FOLDED CORE ABSORBENT ARTICLE

(71) Applicant: Covidien LP, Mansfield, MA (US)

(72) Inventor: Richard Gahan, Wrentham, MA (US)

(21) Appl. No.: 14/713,253

(22) Filed: May 15, 2015

Related U.S. Application Data
(63) Continuation of application No. 14/541,718, filed on Nov. 14, 2014.
(60) Provisional application No. 61/905,441, filed on Nov. 18, 2013.
(60) Provisional application No. 61/904,670, filed on Nov. 15, 2013.

(51) Int. Cl. A61F 13/15

(52) U.S. Cl. CPC A61F 13/15634; A61F 13/15699

(57) ABSTRACT

A liquid absorbent article includes an absorbent core including a liquid absorbent member and a unitary liquid distribution sheet disposed against a surface of the liquid absorbent member. The liquid absorbent member includes a first absorbent layer surface, a second absorbent layer surface, a third absorbent layer surface in contact with the second absorbent layer surface, and a fourth absorbent layer surface. The unitary liquid distribution sheet includes a first distribution layer surface in contact with the first absorbent layer surface of the liquid absorbent member and a second distribution layer surface in contact with the fourth absorbent layer surface of the liquid absorbent member.

### CROSS REFERENCE TO RELATED APPLICATIONS

This application is a continuation of U.S. patent application Ser. No. 14/541,718, titled FOLDED CORE ABSORBENT ARTICLE AND RELATED METHOD, filed on Nov. 14, 2014, which claims the benefit of U.S. Provisional Patent Application No. 61/904,670, titled ABSORBENT ARTICLE WITH WRAP LIQUID DISTRIBUTION LAYER AND RELATED METHODS, filed on Nov. 15, 2013, and U.S. Patent Application No. 61/905,441, titled ABSORBENT ARTICLE WITH WRAP LIQUID DISTRIBUTION LAYER AND RELATED METHODS, filed on Nov. 18, 2013. The entire contents of each of U.S. patent application Ser. No. 14/541,718; U.S. Provisional Patent Application No. 61/904,670; U.S. Provisional Patent Application No. 61/904,670 are incorporated herein by reference for all purposes.

### TECHNICAL FIELD

The present disclosure generally pertains to absorbent articles such as incontinence care articles, and in particular embodiments, relates to adult briefs, diapers, training pants and youth pants having a fluff-free absorbent core with an integrated liquid acquisition distribution layer wrapped around a central portion thereof as well as related methods thereto.

### BACKGROUND

Incontinence care articles such as briefs, diapers, youth pants, and training pants typically have a first liquid permeable layer disposed proximate or even adjacent a skin surface, an outer impermeable layer, and an absorbent core disposed between the first liquid permeable layer and the outer permeable layer.

The absorbent core can include one or more absorbent materials, including a mixture of superabsorbent polymer particles and fluff pulp. Some products have an embossed core that defines diamond-shaped pockets including a mixture of fluff pulp material and superabsorbent polymer particles. The embossing process, however, relies on hydrogen bonding phenomena to create the defined embossing lines.

### SUMMARY

There is disclosed a liquid absorbent article including an absorbent core having a liquid absorbent member, which can be a unitary liquid absorbent member, and a unitary liquid distribution layer, e.g., a unitary liquid distribution sheet, disposed against a surface of the liquid absorbent member. The absorbent core can have an inner section comprised of a first portion of the unitary liquid distribution sheet having a first distribution layer surface. A first portion of the unitary absorbent member can have a first absorbent layer surface in contact with the first distribution layer surface and a second absorbent layer surface. A second portion of the unitary absorbent member can have a third absorbent layer surface in contact with the second absorbent layer surface, and a fourth absorbent layer surface in contact with a second distribution layer surface. The absorbent core can further include a front section and a rear section; the inner section is typically disposed between the front and rear sections. In some embodiments, a surface area of the unitary liquid distribution sheet can be substantially the same as a surface area of the liquid absorbent member. The absorbent article can further include a liquid impermeable back sheet and a permeable top sheet; the absorbent core can be disposed between the top sheet and the back sheet. The liquid absorbent member can include a layer of superabsorbent particles disposed between tissue layers. The first absorbent layer surface can be in fluid communication with the fourth absorbent layer surface through the unitary liquid distribution sheet.

There is disclosed a disposable article having a waist opening and a pair of leg openings. The disposable article is in the form of a chassis with a front portion, a back portion, and a crotch portion disposed between the back portion and the front portion. A liquid absorbent core is at least partially disposed in the crotch portion. The liquid absorbent core includes a unitary liquid distribution sheet disposed on a unitary absorbent member. The liquid absorbent core has an unfolded section and a first folded section. The first folded section includes a first portion of the unitary absorbent member and a first portion of the unitary liquid distribution sheet. The first folded section is disposed on at least a portion of the unfolded section. The liquid absorbent core can further include a second folded section including a second portion of the unitary absorbent member and a second portion of the unitary liquid distribution sheet. The second folded section is disposed on at least a portion of the unfolded section. The unitary liquid distribution sheet can surround the first folded section and the second folded section. In cases, a surface area of the unitary liquid distribution sheet can be substantially the same as a surface area of the liquid absorbent member. The absorbent member can include a layer of superabsorbent particles disposed between layers. The layers include any one of nonwoven, woven, spunbond, or air-laid fibers. The unitary liquid distribution sheet can surround the first folded section and the second folded section.

There is disclosed a method directed to fabricating an absorbent article. The method, in some cases, can include providing a liquid absorbent member including a layer of superabsorbent particles; disposing a liquid distribution sheet on the liquid absorbent member to produce a laminate; creating a first partial cut from a first edge toward a central portion of the laminate; creating a second partial cut from the first edge of the laminate to form a first laminate flap; creating a third partial cut from a second edge toward the central portion of the laminate; creating a fourth partial cut from the second edge of the laminate to form a second laminate flap; and folding the first laminate flap and the second laminate flap onto the central portion of the laminate to produce the absorbent article. The first laminate flap can be a flared flap having a width dimension that diminishes from the first edge towards the central portion and the second laminate flap can be a flared flap having a width dimension that diminishes from the second edge towards the central portion. The first partial cut is a linear cut at a first obtuse angle relative to the first edge of the laminate. The second partial cut is a linear cut at a first acute angle relative to the first edge of the laminate. The third partial cut is a linear cut at a second obtuse angle relative to the second edge of the laminate. The fourth partial cut is a linear cut at a second acute angle relative to the second edge of the laminate. The first obtuse angle is substantially equal to the second obtuse angle and the first acute angle is substantially equal to the second acute angle. Each of the first partial cut, the second partial cut, the third partial cut, and the fourth partial cut can be a curvilinear cut. Respective ends of the first partial cut and the second partial cut define a first fold line. Respective ends of the third partial cut and the fourth partial cut can define a second fold line. Thus, the first laminate flap can be folded along the first fold line and the second laminate flap can be folded along the second fold line. The liquid absorbent member can consist essentially of the layer of superabsorbent particles disposed between layers of any one of nonwoven, woven, spunbond, or air-laid fibers. Folding the first laminate flap onto the central portion and folding the second laminate flap onto the central portion can include butting an edge of the first laminate flap against an edge of the second laminate flap. Folding the first laminate flap onto the central portion and folding the second laminate flap onto the central portion can create a laminated middle portion consisting essentially of the liquid absorbent material enveloped within the liquid distribution sheet.

### DESCRIPTION OF THE DRAWINGS

The features and advantages of the present disclosure will become apparent with regard to the following description, claims, and accompanying figures, wherein:

FIG. 1 is a plan view of an absorbent article having a fluffless, folded absorbent core in accordance with one or more aspects of the present disclosure;

FIG. 2 is a cross-sectional view of a portion of the absorbent article taken along the line 2-2 of FIG. 1;

FIG. 3 is a cross-sectional view of the absorbent article taken along the line 3-3 of FIG. 1;

FIG. 4 is a plan view illustrating a laminated member that may be utilized to create an absorbent core of the absorbent article in accordance with one or more embodiments of the present disclosure;

FIG. 5 is a cross-sectional view of the laminated member taken along lines 4-4 of FIG. 4; and

FIG. 6 is a plan view of an alternate embodiment.

### DESCRIPTION

The presently disclosed absorbent articles can include a substrate upon and within which fluid discharge, e.g., liquid insult, may be applied and absorbed. For illustrative purposes, the absorbent articles in accordance with the present disclosure will be exemplarily described as a diaper; however, one or more of the features and aspects disclosed herein may be directed or implemented in various liquid absorbent products such as feminine hygiene products such as menstrual pads, adult incontinence products such as diapers, youth pants, training pants, adult briefs, protective underwear, pads and bladder control pads, pet training pads, and other disposable products utilized to absorb fluids.

Absorbent articles can include a multi-layer arrangement with a permeable top sheet for engaging the body surface, a fluid impermeable back sheet for preventing fluid leakage through the article, and an absorbent core disposed therebetween. One or more aspects of the presently disclosed articles and related methods involve a preformed absorbent core. In some particular configurations, the absorbent articles utilize a preformed, fluff-free or fluffless absorbent core. In embodiments directed to the diapers, training pants, youth pants, briefs, and incontinence products as well as absorbent pads, the absorbent article further has a liquid impermeable back sheet and a permeable top sheet, wherein the absorbent core is disposed between the top sheet and the back sheet.

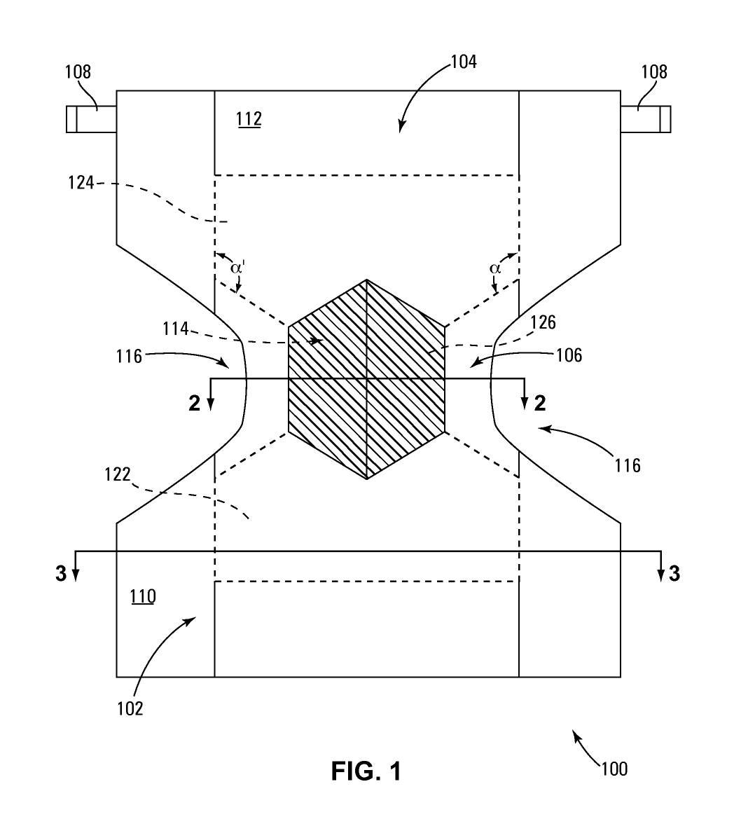

In an embodiment as exemplarily illustrated in FIG. 1, the absorbent article 100 is disclosed in the form of a diaper. The absorbent article 100 is generally formable into a chassis having a front waist portion 102, a back waist portion 104, and a crotch portion 106 disposed between the front and back waist portions 102, 104. The front waist portion 102 and/or the back waist portion 104 may include a pair of fastening tabs or tapes 108 for securing the respective front and back waist portions 102, 104 about the user.

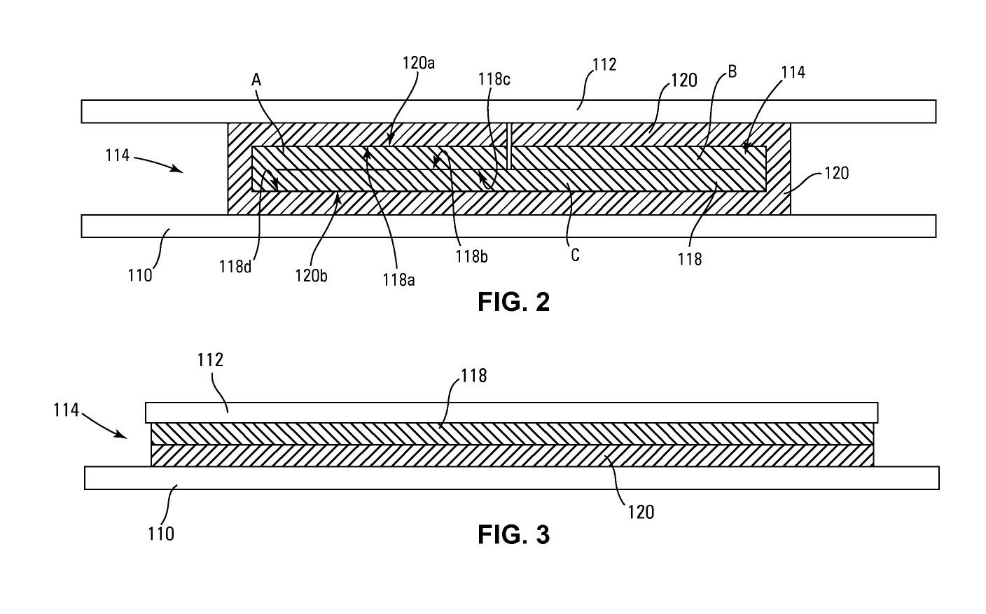

With reference to FIGS. 2 and 3, in conjunction with FIG. 1, the absorbent article 100 further includes a liquid impermeable back sheet 110, a permeable top sheet 112, e.g. a liquid permeable top sheet, and an absorbent core 114 disposed between the liquid impermeable back sheet 110 and the permeable top sheet 112. The back sheet 100 and the top sheet 112 may or may not be coextensive in size and shape.

The back sheet 110 can be a fluid impervious layer for preventing liquid absorbed and contained in the absorbent core 114 from wetting articles which contact the underpad, such as, but not limited to, undergarments, pants, pajamas, and bed sheets. The back sheet 110 may be: a woven material; a non-woven material; a liquid-impervious fabric; a cellulosic film; a polymeric film such as a thermoplastic film of polyethylene or polypropylene; an impregnated fluid repellent paper; a composite material, e.g., a polylaminate, such as a film-coated non-woven material; or combinations thereof. The back sheet 110, or at least portions thereof, may be embossed or may be matte-finished to provide a cloth-like appearance, and/or colored for ready identification. The back sheet 110 may also be breathable to allow at least some vapors to escape or pass from the absorbent core 114, while preventing fluid discharge from passing therethrough. Thus, in some cases, the back sheet 100 can be liquid impermeable but can be a vapor permeable back sheet.

The top sheet 112 can be a woven or nonwoven fabric including polymeric fibers. In some cases, the top sheet 112 can be formed of one or more bicomponent polymeric fibers. For example, the top sheet 112 can be a fabric with first bicomponent fibers woven with second bicomponent fibers. The top sheet 112 is often a fluid pervious layer for permitting liquid, e.g., menses or urine, to penetrate readily through its thickness. The top sheet 112 may be compliant and/or soft to the touch so that it does not irritate skin. The top sheet 112 may be manufactured from a wide range of materials such as woven and non-woven materials, e.g., a non-woven web of fibers; polymeric materials such as thermoplastic films having apertures, plastic films having apertures, and hydro-formed thermoplastic films; porous foams; reticulated foams; reticulated thermoplastic films; and thermoplastic scrims. Suitable fibers that may be utilized to construct woven and non-woven materials include, for example, natural fibers, e.g., wood or cotton fibers, synthetic fibers, e.g., polymeric fibers such as polyester, polypropylene, or polyethylene fibers, and combinations of natural and synthetic fibers.

In embodiments in which the top sheet 112 is a non-woven web, the web may be spun-bonded, carded, wet-laid, melt-blown, hydro-entangled, or formed using any method suitable for forming a non-woven web. In accordance with further embodiments of the disclosure, at least a portion of the liquid pervious top sheet can be formed of a nonwoven fibrous layer of polyolefinic fibers. The fibers can be multicomponent fibers. At least a portion of the fibers of at least a portion of top sheet 112 can include bicomponent fibers having a first component that has a first fusion point and a second component that has a second fusion point that is less than the first fusion point. In accordance with still further aspects of the disclosure, at least a portion of the top sheet can include thermobondable polymeric bicomponent fibers including a high-melting point core and a low melting point sheath substantially surrounding the core. The top sheet 120 can include at least about 50% by weight of bicomponent fibers. Thus, for example, the top sheet can include monolithic fibers formed of a polyolefin which, in some cases, can be the same polyolefin as the core. In embodiments, however, the top sheet 112 includes bicomponent fibers. In other embodiments, the top sheet 112 consists essentially of bicomponent fibers. In still other embodiments, top sheet 112 consists of bicomponent fibers.

The top sheet 112 can thus include bicomponent fibers having a core of a first polyolefin and a sheath around the core of a second polyolefin. The top sheet 112 can also consist essentially of bicomponent fibers having a core of a first polyolefin and a sheath around the core of a second polyolefin. In other embodiments, the top sheet 112 consists of bicomponent fibers having a core of a first polyolefin and a sheath around the core of a second polyolefin.

In some configurations, the top sheet 112 can include a plurality of layers. For example, the top sheet 112 can include a first layer, as the body side layer, including bicomponent fibers and a second layer, distal to the body side layer, including monocomponent fibers. In some configurations of the top sheet, the second layer can include spun bond monocomponent fibers of polyolefin, which may be a polyethylene, and can be the same polyethylene of the sheath of the bicomponent fibers of the first layer. In other embodiments, the top sheet 112 includes a first layer, preferably as the body side layer, consisting of or consisting essentially of bicomponent fibers, and a second layer, typically a distal second layer, including or consisting of monocomponent fibers. In some particular configurations of the top sheet, the second layer can consist of spun bond monocomponent fibers of polyolefin, in some cases a polyethylene, and can be the same polyethylene of the sheath of the bicomponent fibers of the first layer.

In other embodiments, the first polyolefin can be a polypropylene and the second polyolefin can be polyethylene. In other configurations, the first polyolefin can be a high-density polyethylene and the second polyolefin can be low-density polyethylene. For example, the sheath can include a linear low-density polyethylene having a density of less than or about 0.95 g/cm³. The core can include a high-density polyethylene having a density of greater than 0.95 g/cm³.

In one embodiment, the back and top sheets 110, 112 are coextensive lengthwise of the article 100, while the width of the top sheet 112 is less than the width of the back sheet 110 in the front and back waist portions 102, 104 of the article. The back and/or top sheets 110, 112 may define a pair of side edges 116. Each side edge 116 includes a central, cutout to define a respective leg cut out. The crotch portion 106 is located between the leg cutouts. The back sheet 110 and/or the top sheet 112 can be any suitable shape and dimensions for other designs or constructions, as will be clear to those skilled in the art.

The back sheet 110 may be bonded to the top sheet 112 around its entire periphery, with the absorbent core 114 interposed therebetween. The back sheet 110 and top sheet 112 can be joined together in any suitable manner, e.g., by adhesive bonding. The adhesives can be applied in any manner such as by spraying, slot-coat extrusion, and printing. The applied adhesive can be in any desired configuration or design, such as continuous or discontinuous beads, continuous or discontinuous swirls, meltblown patterns, and other predefined patterns. Alternatively, the joining of layers and structures can be accomplished by heat sealing or by ultrasonic bonding.

Referring still to FIGS. 1-3, the absorbent core 114 of the absorbent article 100 will be described. The absorbent core 114 includes at least one liquid absorbent member 118 and a unitary liquid distribution sheet 120 disposed against and/or adhered to a surface of the at least one liquid absorbent member 118. In certain embodiments, the absorbent core 114 is formed as a laminate of the absorbent member 118 and the distribution sheet 120. In some cases, the absorbent core can consist essentially of the absorbent member 118 and the distribution sheet 120. As depicted in FIG. 1, the absorbent core 114 can have a front section 122, a rear section 124, and an inner section 126 disposed between the front and rear sections 122, 124. The front and rear sections 122, 124 of the absorbent member 118 may extend at least partially into respective front and back waist portions 102, 104 of the absorbent article 100. The inner section 126 of the absorbent core 114 may at least partially encompass the crotch portion 106 of the absorbent article 100. In certain embodiments, the absorbent core 114 may be devoid of the front and rear sections 122, 124, and include only the inner section 126. The front and rear sections 122, 124 of the absorbent core 114 may include the single laminate with the absorbent member 118 superposed relative to the distribution sheet 120 as depicted in the cross-sectional view of FIG. 3.

With reference again to the cross-sectional view of FIG. 2, in conjunction with FIG. 1, the inner section 126 of the absorbent core 114 is folded onto itself to provide a multilayer fluid-acquisition unit. The acquisition unit effectively transfers and contains body fluids from the subject, and is adapted to accommodate repeated insults of body fluids. The details of forming the folded inner section will be described herein below. In one embodiment, the inner section 126 of the absorbent core 114 is folded such that the liquid distribution sheet 120 at least partially or fully encompasses the liquid absorbent member 118. For example, the inner section 126 of the absorbent core 114c may include first and second folded sections A, B and an unfolded section C upon which the first and second folded sections A, B are folded. In this manner, the inner section 126 includes two layers of the absorbent member 118 surrounded by the liquid distribution sheet 120. The first and second folded sections A, B may or may not be in juxtaposed relation.

In the embodiment of FIG. 2, the folded inner section 126 provides a first portion having a first distribution layer surface 120a of the liquid distribution sheet 120, a first absorbent layer surface 118a of the absorbent member 118 in contact, e.g., in some cases, in direct contact, with the first distribution layer surface 120a and a second absorbent layer surface 118b opposing the first absorbent layer surface 118a. The folded inner section 126 also provides a second portion having a third absorbent layer surface 118c of the unfolded section C in contact, e.g., in some cases, in direct contact, with the second absorbent layer surface 118b, and a fourth opposing absorbent layer surface 118d in contact, e.g., in some cases, in direct contact, with a second distribution layer surface 120b of the unitary liquid distribution sheet 120. Thus, in some advantageous configurations, the first absorbent layer surface 120a is in fluid communication with the fourth absorbent layer surface 120d through the unitary liquid distribution sheet 120.

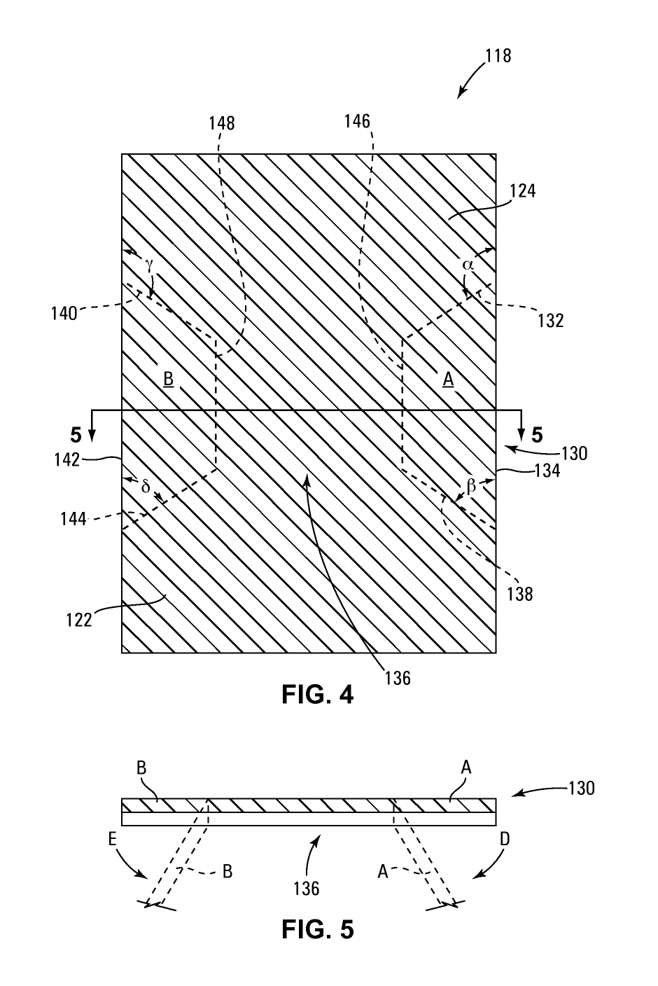

FIGS. 4-5 illustrate the absorbent core 114 prior to folding or creating the folded inner section 126 between the front and rear sections 122, 124. In some embodiments, the liquid distribution sheet 120 and the absorbent member 118 are coextensive, e.g., a surface area of the unitary liquid distribution sheet 120 can be substantially the same as a surface area of the liquid absorbent member 118. As exemplarily illustrated, the respective contacting surfaces of the liquid absorbent member 118 and the liquid distribution sheet 120 have substantially the same surface areas.

The method of formation of the absorbent core followed by a method of fabricating an absorbent article, such as, for example, absorbent article 100 described above will now be described. With continued reference to FIGS. 4-5, the method includes disposing the liquid distribution sheet 120 on the liquid absorbent member 118, which includes superabsorbent material, e.g., a layer of superabsorbent particles, to produce a laminate 130. A first partial cut 132 is created from a first edge 134 of the liquid absorbent core 140, toward a central portion 136 of the laminate 130. A second partial cut 138 is created from the first edge 134 of the laminate 130 to form a first laminate flap or first folded section A, as mentioned above. A third partial cut 140 is created from a second edge 142 of laminate 130 toward the central portion 136 of the laminate 130. A fourth partial cut 144 is created from the second edge 142 of the laminate 130 to form a second laminate flap or second folded section B, as mentioned above. The first laminate flap A and the second laminate flap B are both folded, in the direction indicated by arrows D and E in FIG. 5, and as depicted in phantom onto the central portion 136 of the laminate 130 to produce the absorbent core 114 shown in FIGS. 1 and 2.

The first laminate flap A can be a flared flap having a width dimension that diminishes from the first edge 134 towards the central portion 136 and the second laminate flap B can be a flared flap having a width dimension that diminishes from the second edge 142 towards the central portion 136.

The respective ends of the first partial cut 132 and the second partial cut 138 can define a first fold line 146 (FIG. 4) and respective ends of the third partial cut 140 and the fourth partial cut 144 can define a second fold line 148 (FIG. 4). Thus, the first laminate flap A can be folded along the first fold line 146 and the second laminate flap B can be folded along the second fold line 148 to create the liquid absorbent core 114. The fold lines 146, 148 may be perforated, cut or scored, or alternatively, devoid of any cuts. Thus, in some cases, folding the first laminate flap A and the second laminate flap B onto the central portion 136 can involve butting an edge of the first laminate flap A against an edge of the second laminate flap B.

The first partial cut 132 can be a linear cut at a first obtuse angle α relative to the first edge 134 of the laminate 130. The second partial cut 138 can be a linear cut at a first acute angle β relative to the first edge 134 of the laminate 130. The third partial cut 140 can be a linear cut at a second obtuse γ angle relative to the second edge 142 of the laminate 130. The fourth partial cut 144 can be a linear cut at a second acute angle δ relative to the second edge 142 of the laminate 130. In some embodiments, for example, as illustrated in FIG. 4, the first obtuse angle α is substantially equal to the second obtuse angle α′ and the first acute angle β can be substantially equal to the second acute angle β′.

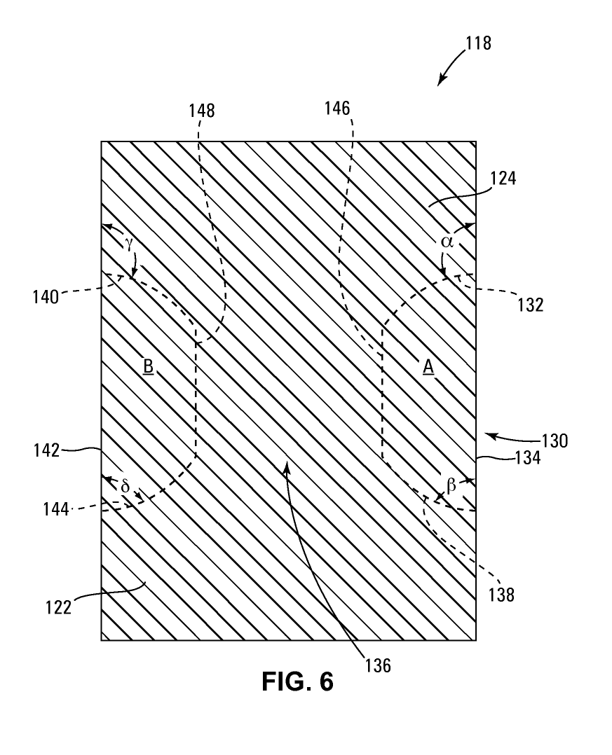

Each of the first partial cut 132, the second partial cut 138, the third partial cut 140, and the fourth partial cut 144 may be a linear cut, as exemplarily illustrated in FIG. 4, or any one or more of such partial cuts 132, 138, 140, 144 can be a non-linear or curvilinear cut, as depicted in FIG. 6.

In some advantageous configurations, the liquid absorbent member consists essentially of the layer of superabsorbent particles disposed between layers. The layers between which the superabsorbent particles are disposed can include any one of nonwoven, woven, spunbond, or air-laid fibers. Suitable materials for use as the super absorbent polymer particles include starch type, starch-graft polymers, cellulosic, and synthetic types, starch-acrylic acid (salt) graft copolymers, saponified starch-acrylonitrile copolymers, cross-linked sodium carboxymethyl cellulose, acrylic acid (salt) polymers, cross-linked polyacrylate polymers, polyacrylamides, polyethylene oxides, polyvinyl alcohols, polysuccinimides, hydrolyzed polyacrylonitriles, combinations thereof. As a shape for the super absorbent polymer particles, powder-particle may be desirable, but other shapes can be also used.

In some advantageous configurations, folding the first laminate flap A and the second laminate flap B onto the central portion 136 provides a laminated middle portion consisting essentially of the liquid absorbent member 118 enveloped within the liquid distribution sheet 120. Thus, in some cases, the absorbent media can be spread over a larger area, resulting in an absorbent incontinence article that is thinner and more comfortable to the user while providing protection in crotch area, instead of using a thick core strip limited by crotch width and further reduces waste material by cutting out the leg opening from the crotch area.

A sample was made by cutting a core out of a GELOK® 11040-72-S/S absorbent material. This absorbent material contained superabsorbent polymer at 110 gsm basis weight with 12 tissues. The absorbent material was cut to be 14 inch wide and 24 inch length for 335 in², at 48 gram. An 11 inch long by 14 inch wide piece of 15 gsm hydrophilic spunbond liquid acquisition distribution sheet (from Polymer Group, Inc.) was attached to one side at approximately 7 inch from one end and approximately 6 inch from the other end of the GELOK® material to produce a laminate. Diagonal cuts were made at about 11 inch from a back end and at about 6 inch from a front end to form flaps. The flaps were folded over to meet in the middle to make an absorbent core with a 7 inch wide crotch area. The absorbent core was attached to polyfilm with the crotch area centered along the elasticized leg openings thereof and a top sheet was attached thereto. This brief weighed about 73 grams with a core having a thickness (at the crotch) of about 0.90 inch and about 0.040 inch at the remainder. The absorbency capacity is expected to be about 1800 grams of saline.

Having described the present disclosure in detail, it will be apparent that modifications and variations are possible without departing from the scope of the present disclosure defined in the appended claims. For example, different types of superabsorbent material can be used to tailor the performance characteristics of the absorbent core or the absorbent article, at various relative amounts and at various regions of the core or the absorbent article.

When introducing elements of the present disclosure or the embodiments(s) thereof, the articles “a,” “an,” “the” and “said” are intended to mean that there are one or more of the elements. The terms “comprising,” “including” and “having” are intended to be inclusive and mean that there may be additional elements other than the listed elements. While the above description refers to many devices, methods, and/or elements as including or having certain features and/or components, it is to be understood that disclosure also encompasses devices, methods, and/or elements “consisting essentially of” or “consisting of” those features and/or components.

Thus, the transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristics of the claimed invention. A “consisting essentially of” claim occupies a middle ground between closed claims that are written in a “consisting of” format and fully open claims that are drafted in a “comprising” format.

While the above description contains many specifics, these specifics should not be construed as limitations on the scope of the present disclosure, but merely as exemplifications of embodiments thereof. It is envisioned that the elements and features illustrated or described in connection with one exemplary embodiment may be combined with the elements and features of another exemplary embodiment without departing from the scope of the present disclosure, and that such modifications and variations are also intended to be included within the scope of the present disclosure. Those skilled in the art will envision many other possible variations that are within the scope and spirit of the present disclosure.

What is claimed is:

1. A method of fabricating an absorbent article comprising:
providing a liquid absorbent member including a layer of superabsorbent particles;
disposing a liquid distribution sheet on the liquid absorbent member to produce a laminate;
creating a first partial cut from a first edge toward a central portion of the laminate;
creating a second partial cut from the first edge of the laminate to form a first laminate flap;
creating a third partial cut from a second edge toward the central portion of the laminate;
creating a fourth partial cut from the second edge of the laminate to form a second laminate flap; and
folding the first laminate flap and the second laminate flap onto the central portion of the laminate to produce an absorbent core of the absorbent article.

2. The method of claim 1, wherein the first laminate flap is a flared flap having a width dimension that diminishes from the first edge towards the central portion, and wherein the second laminate flap is a flared flap having a width dimension that diminishes from the second edge towards the central portion.

3. The method of claim 1, wherein the first partial cut is a linear cut at a first obtuse angle relative to the first edge of the laminate, the second partial cut is a linear cut at a first acute angle relative to the first edge of the laminate, the third partial cut is a linear cut at a second obtuse angle relative to the second edge of the laminate, the fourth partial cut is a linear cut at a second acute angle relative to the second edge of the laminate, and wherein the first obtuse angle is substantially equal to the second obtuse angle and the first acute angle is substantially equal to the second acute angle.

4. The method of claim 1, wherein each of the first partial cut, the second partial cut, the third partial cut, and the fourth partial cut is a curvilinear cut.

5. The method of claim 1, wherein respective ends of the first and second partial cuts define a first fold line and respective ends of the third and fourth partial cuts define a second fold line, and wherein the first laminate flap is folded along the first fold line and the second laminate flap is folded along the second fold line.

6. The method of claim 1, wherein the liquid absorbent member consists essentially of the layer of superabsorbent particles disposed between layers of any one of nonwoven, woven, spunbond, or air-laid fibers.

7. The method of claim 1, wherein folding the first laminate flap and the second laminate flap onto the central portion includes butting an edge of the first laminate flap against an edge of the second laminate flap.

8. The method of claim 1, wherein folding the first laminate flap and the second laminate flap onto the central portion provides a laminated middle portion consisting essentially of the liquid absorbent member enveloped within the liquid distribution sheet.
